(19) United States
(12) Patent Application Publication
(10) Pub. No.: US 2017/0296122 A1
(43) Pub. Date: Oct. 19, 2017

(54) DEVICE TO HOLD MULTIPLE BLOOD PRESSURE READING UNITS WITH POSITION INDICATOR

(71) Applicant: Abbas M HUSAIN, Voorhees, NJ (US)

(72) Inventor: Abbas M HUSAIN, Voorhees, NJ (US)

(21) Appl. No.: 15/636,291

(22) Filed: Jun. 28, 2017

Related U.S. Application Data
(63) Continuation-in-part of application No. 14/957,404, filed on Dec. 2, 2015.

(51) Int. Cl. A61B 5/00; A61B 5/022

(52) U.S. Cl. CPC A61B 5/684; A61B 5/02233; A61B 5/742

(57) ABSTRACT

A device to hold multiple blood pressure reading units with position indicator has a blood pressure monitor holder with a shelf portion holding multiple blood pressure monitors. The holder is moveably attached using a position indicator attached to the patient using a collar. The position indicator is a flexible ruler that shows the position of the units below the collar. The position indicator attaches to the comfortable adjustable collar that attaches to a patient's neck and is retractably positioned to indicate the position of the reading to be taken. If there is a significant difference between the left and right readings, then further tests are ordered. This establishes a repeatable baseline where further readings are taken from the same position. The location of the reading can provide important diagnosis information at the primary care level long before symptoms and complications begin.

### CROSS REFERENCE TO RELATED APPLICATIONS

This is a continuation in part of U.S. patent application Ser. No. 14/957,404, filed Dec. 2, 2015 the entire content of which is hereby incorporated by reference.

### BACKGROUND OF THE INVENTION

Blood pressure is an important part of a medical workup that provides medical professionals with information regarding the underlying health of a patient. Recent studies have suggested that taking the blood pressure simultaneously on both the left and right side of a patient can provide important information regarding heart disease or other cardiac problems. In an article published in THE LANCET, it was reported that differences between a patient's blood pressure from each arm could help identify patients who could be at risk for heart problems (The Lancet, Volume 379, Issue 9819, Pages 905-914, 10 Mar. 2012; also from the Harvard Medical School http://www.health.harvard.edu/blog/different-blood-pressure-in-right-and-left-arms-could-signal-trouble-201202014174). It is well known that when a patient has a great differential blood pressure bilaterally, it is likely a sign of some serious vascular problem such as aneurysm or constriction of aorta or other vascular problem. If a medical professional wants this information, they have to repeat the blood pressure test on each side which is time consuming and also can lead to inaccuracy because of the time differential between readings.

There is a need for a device that allows a user to place readily available blood pressure measuring devices at specific locations with reference to a patient's body and to record that position for future reference when repeating the measurements.

### SUMMARY OF THE INVENTION

A device to hold multiple blood pressure reading units with position indicator has a blood pressure monitor holder with a shelf portion holding multiple blood pressure monitors. The holder is moveably attached using a position indicator attached to the patient using a collar. The position indicator is a flexible ruler that shows the position of the units below the collar. The position indicator attaches to the comfortable adjustable collar that attaches to a patient's neck and is retractably positioned to indicate the position of the reading to be taken. If there is a significant difference between the left and right readings, then further tests are ordered. This establishes a repeatable baseline where further readings are taken from the same position. The location of the reading can provide important diagnosis information at the primary care level long before symptoms and complications begin.

Other features and advantages of the instant invention will become apparent from the following description of the invention which refers to the accompanying drawings.

### BRIEF DESCRIPTION OF THE DRAWINGS

FIG. 1 is an illustration of a simultaneous bi-lateral blood pressure system with position indicator according to an embodiment of the invention.

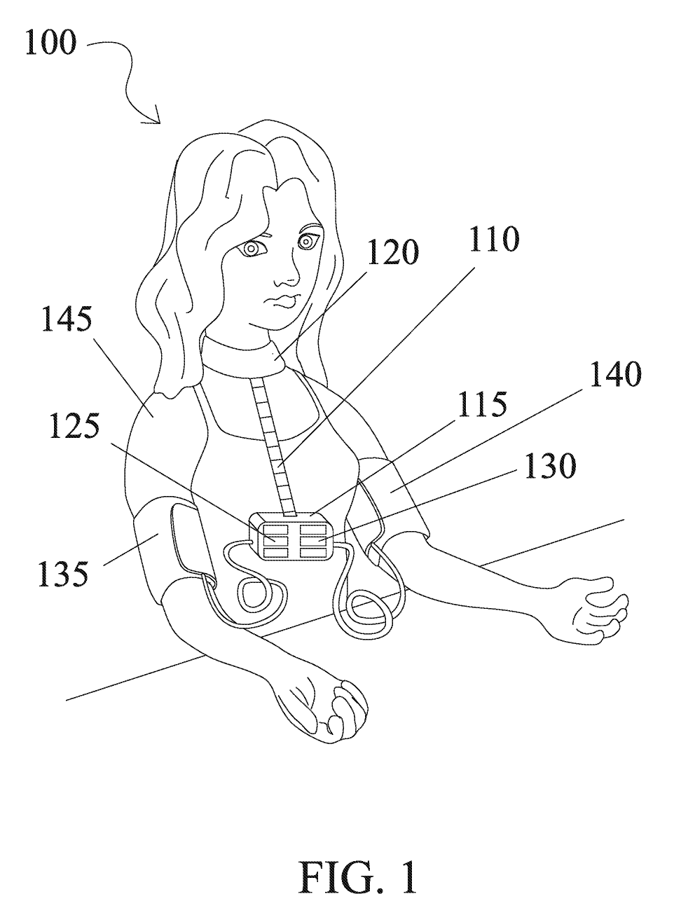

FIG. 2 is an illustration of the simultaneous bi-lateral blood pressure system with position indicator at an alternative blood pressure location.

FIG. 3 is an illustration of the simultaneous bi-lateral blood pressure system with position indicator at another alternative blood pressure location.

FIG. 4 is an illustration of the simultaneous bi-lateral blood pressure system with position indicator at yet another alternative blood pressure location.

FIG. 5 is an illustration of a bi-lateral blood pressure system with position indicator for simultaneous readings using standard monitors according to an embodiment of the invention.

FIG. 6 is an illustration of a bi-lateral blood pressure system with position indicator for simultaneous readings using standard monitors at an alternative blood pressure location.

FIG. 7 is a close up perspective drawing of a blood pressure monitor holder shown in FIG. 5.

FIG. 8 is a side view of the blood pressure monitor holder shown in FIG. 6.

FIG. 9 is a close up perspective drawing of a blood pressure monitor holder shown in FIG. 5 with digital measurement indicator.

FIG. 10 is a close perspective drawing of the blood pressure monitor holder with a unit retainer.

### DETAILED DESCRIPTION OF THE INVENTION

In the following detailed description of the invention, reference is made to the drawings in which reference numerals refer to like elements, and which are intended to show by way of illustration specific embodiments in which the invention may be practiced. It is understood that other embodiments may be utilized and that structural changes may be made without departing from the scope and spirit of the invention.

Referring to the figures, a simultaneous bi-lateral blood pressure system with position indicator 100 is shown having a dual input blood pressure monitor 115 which has a position indicator 110 built in order to allow a user to record the approximate location where the bi-lateral blood pressure measurements are being taken. This can provide a more detailed baseline as well as helping diagnosis other medical issues that might need to be explored if the readings suggest a problem. Position indicator 110 is flexible and rests against a patient 145 and is spring biased to retract and is contained within blood pressure monitor 115. Retracting systems are well known.

A measurement anchoring collar 120 is used to provide a solid pulling point for position indicator 110. Measurement anchoring collar is made of a soft material and is affixed using a hook and loop connector (not shown) to allow it to adjust to the patient's neck. Distance indicator lines are provided on position indicator 110 to allow the user to record the approximate position with respect to patient's body in order to provide consistent readings and to rule out other cause for differential readings. This is helpful where a user wants to track blood pressure over time by knowing where the readings are being taken from and to take all future readings from the same position. Of course other means may be used to allow position indicator 110 to function such as, but not limited to a pin, clip-on mount, lanyard, etc. as long as the end of position indicator 110 is held in place while moving blood pressure monitor 115 to the desired position for the readings to be taken. A single activator (not shown) is used to simultaneously energize both sides to provide simultaneous bi-lateral readings.

A left blood pressure cuff 140 is used to take the reading on the left side and a right cuff 135 is used for the right side. The cuffs (140 and 135) are attached using a hook and loop fastener and are connected to blood pressure monitor 115 with tubing as is known in the art.

Blood pressure monitor 115 has a left display 130 and a right display 125. Both displays (130 and 125) are used to display blood pressure information as well as pulse. Displays 130 and 125 display patient's vital signs. Both the diastolic and systolic pressures are indicated. The instant device allows the health professional to simultaneously take and compare the left side readings with the right side readings and if there is a significant differential, then further tests such as angiogram, CT scan, MRI or MRA or other tests as is known in the art. Individuals with diabetes, smokers or people with genetic predisposition for blood flow constriction are of particular concern. The instant invention can discover these issues before other symptoms become evident thus making the instant invention extremely valuable for quick, easy and inexpensive diagnostic procedures.

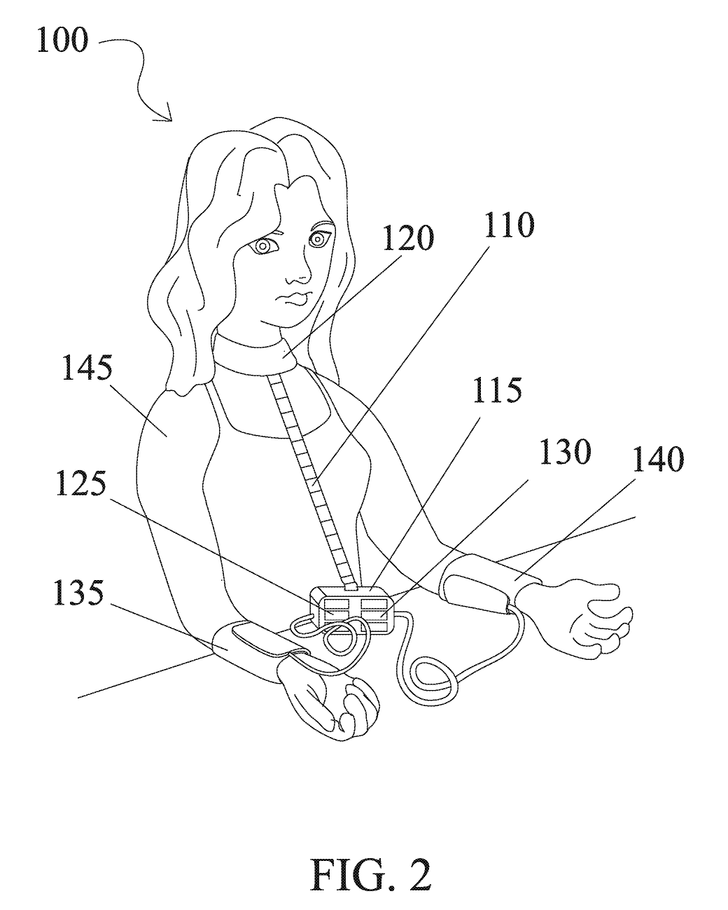

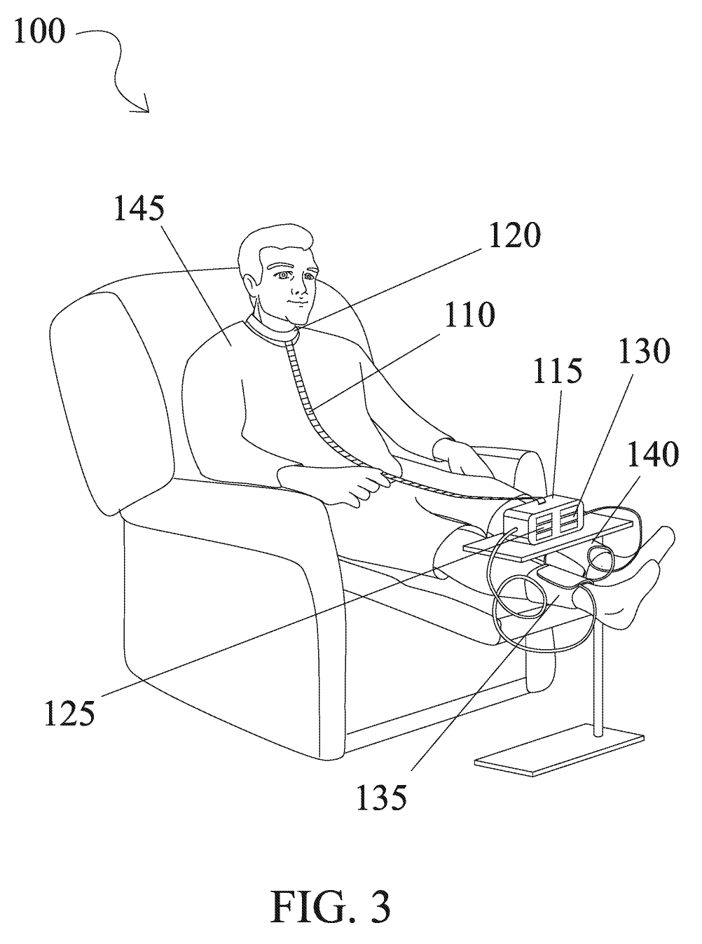

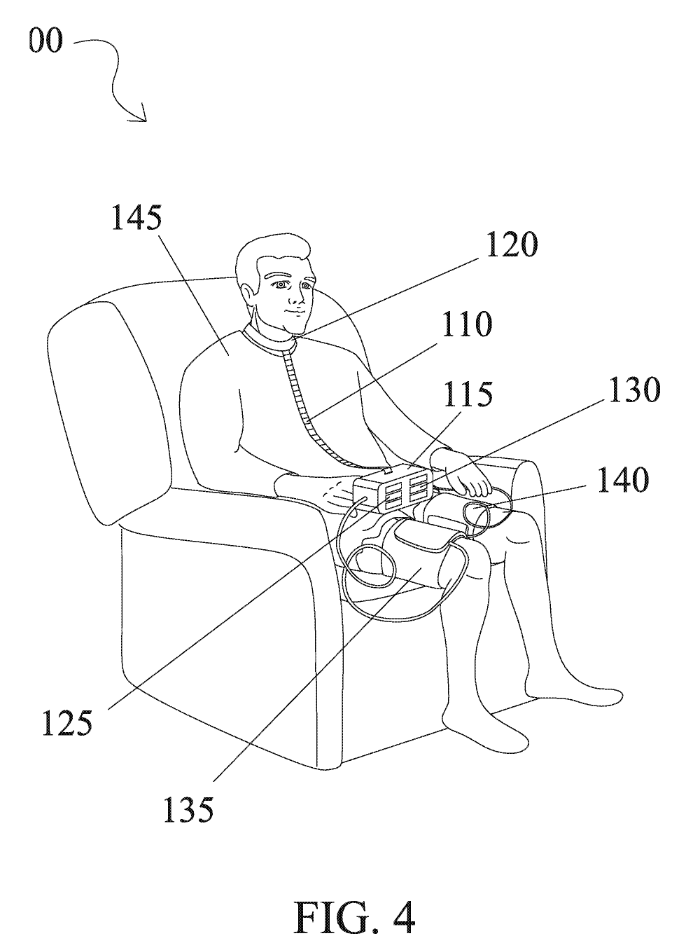

FIGS. 2, 3 and 4 shows blood pressure cuffs 140 and 135 located on the wrists rather than the upper arm. FIGS. 3 and 4 illustrate taking the readings on the lower extremities. Of course the position where the readings are taken can vary and the positions shown are merely for illustrative purposes only and not meant to limit the positions to only the 4 locations shown.

Additionally, although blood pressure monitor is shown having left and right displays (130 and 125) built in, it is known to wirelessly connect to a remote display and although not shown, any display function is acceptable as long as 2 simultaneous blood pressure readings are obtained. Also, although position indicator 110 is shown as being a tape measure, the position could be electronically achieved for example; by using an infra-red positioning device with a transmitter attached to the patient's neck and blood pressure monitor 115 receiving the signal where it is interpreted as distance. Additionally, a digital display 150 may be used to graphically display position or any other information that is desired. Dual input blood pressure monitor 115 may be battery operated or plug into an AC outlet.

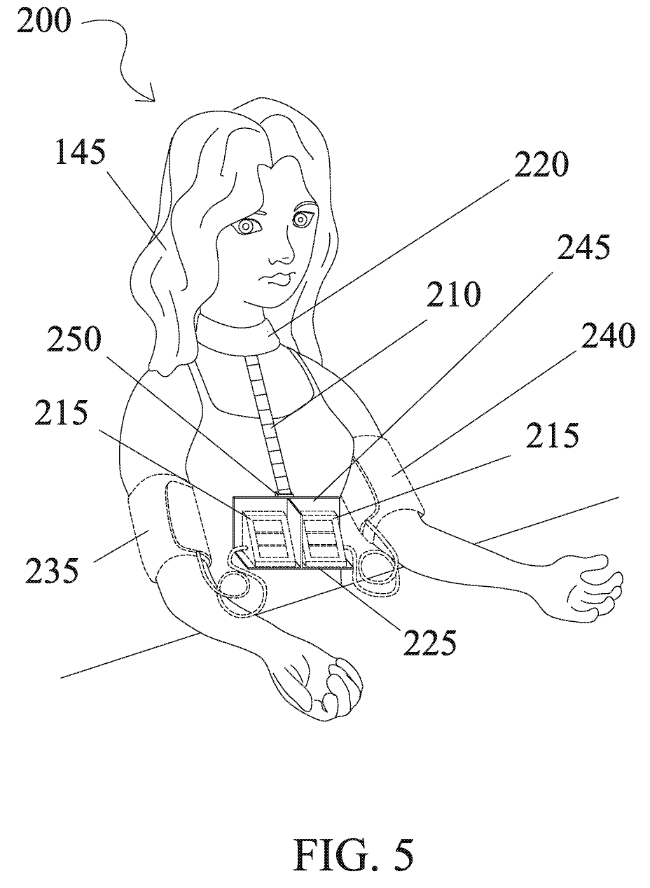

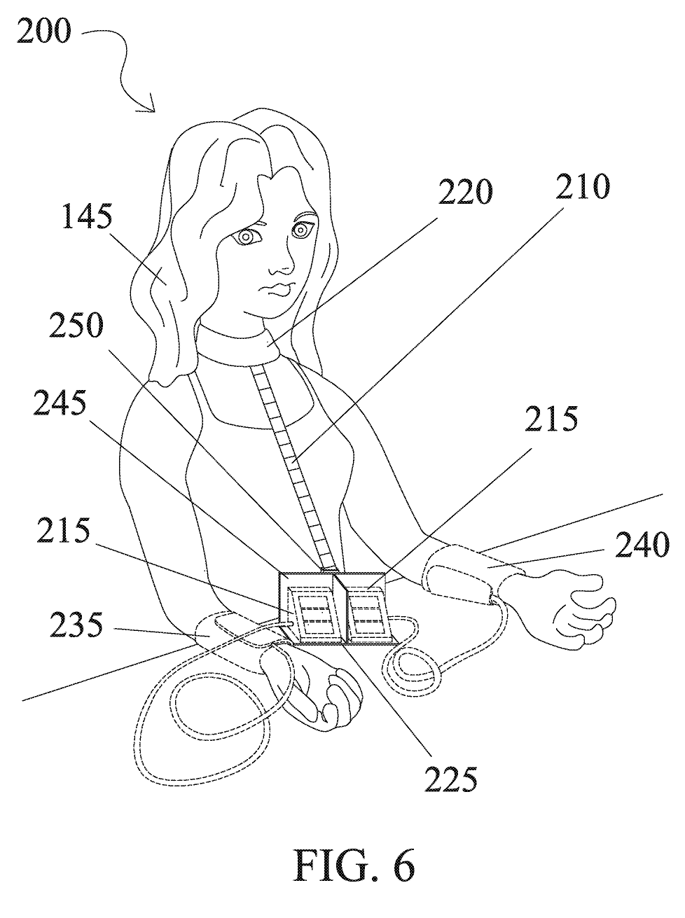

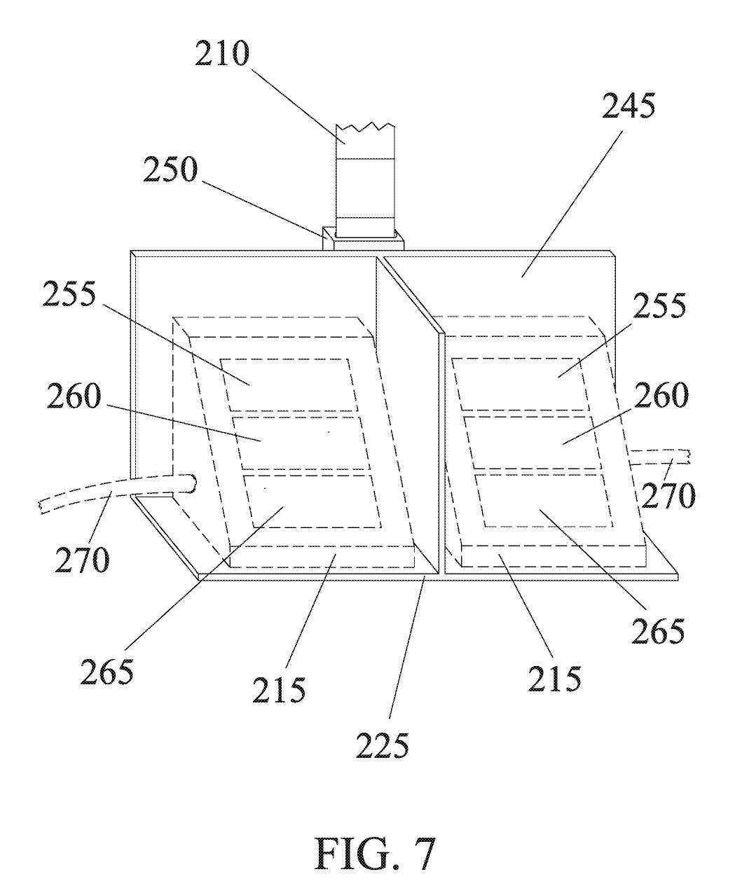

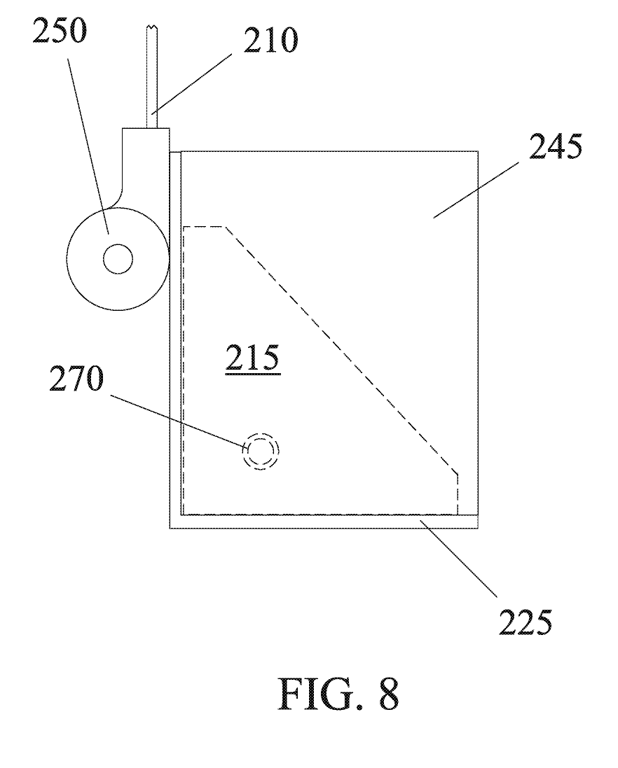

Now referring to FIGS. 5 through 8, a bi-lateral blood pressure system with position indicator for simultaneous readings using standard blood pressure monitors 200 is shown attached to patient 145 using a measurement anchoring collar 220. A position indicator 210 is attached to a position indicator reel holder 250. Position indicator 210 is retractably held within reel holder 250. Reel holder 250 is attached to a blood pressure monitor holder 245 and allows the user to accurately record a specific baseline for taking blood pressure readings. In this way, repeated measurements can be taken at the same position on patient's body. This allows for more reliable readings and gives the user valuable information not easily obtained otherwise.

Blood pressure monitor holder 245 allows the user to place standard “off the shelf” blood pressure monitors 215 on a shelf portion 225. Although blood pressure monitors 215 are shown and depicted as triangular units, it should be clear that any digital blood pressure monitor may be used as long as it can fit on shelf portion 225. This allows the user to easily take simultaneous blood pressure readings from both sides of the patient's body to better diagnose and screen for possible or known health problems. Blood pressure monitors 215 include a left blood pressure cuff 240 and a right blood pressure cuff 235 and pressure tubes 270 as is known in the art. Blood pressure monitors 215 generally includes three displays, a systolic pressure display 255, a diastolic pressure display 260 and a pulse rate display 265.

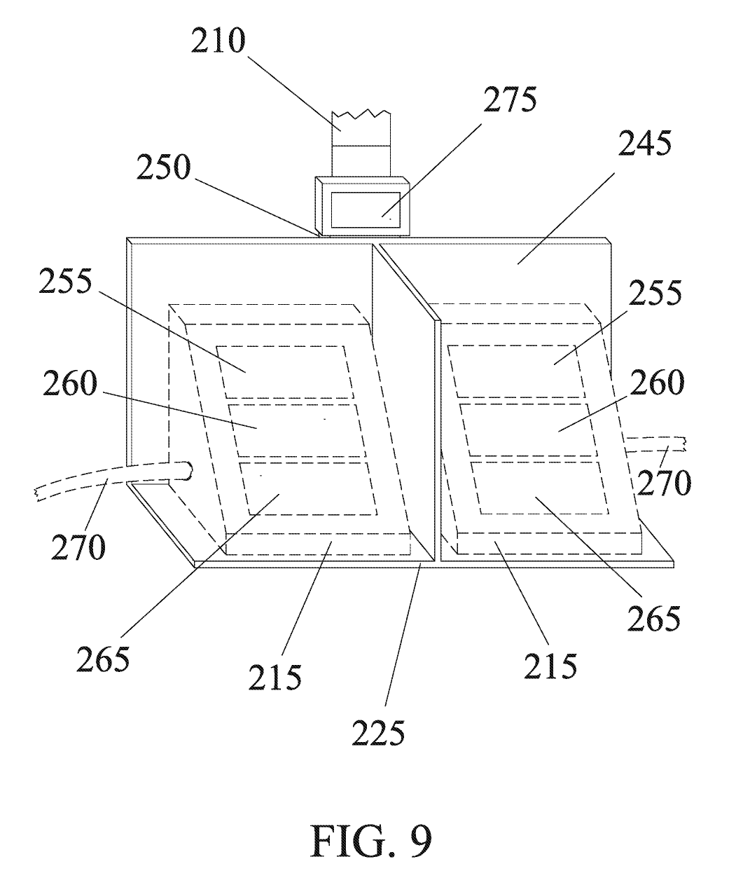

Referring to FIG. 9, a digital measurement display 275 is shown to display the position of the blood pressure units to allow the user to repeat blood pressure measurements from a repeatable position as described above.

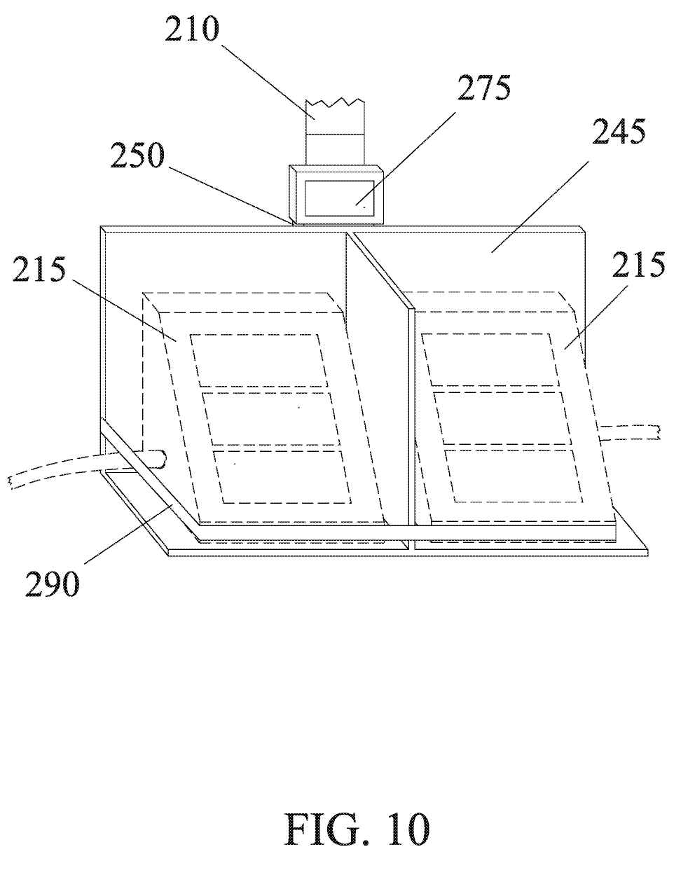

Now referring to FIG. 10, blood pressure monitor holder 245 is shown having a unit retainer 280 that is used to secure blood pressure monitors 215 in place during use. Unit retainer 280 is an elastic strap that removably secures monitors in place. Of course other means may be used to secure monitors in place such as, but not limited to hook and loop straps, clips, etc. as long as the units are removably held in place during use. In use, the units should be placed at the level of the cuffs that are used to take the measurements and the position is noted. Later more readings are taken by placing the units at the same location.

Various pathologies such as constriction, dilation or obstruction occur in the middle of the aorta for the upper extremities or in the artery going down to the legs. For the upper extremities, there is a common arch of aorta radiating from the heart with one radial artery providing blood to the right arm and another radial artery for the left arm. If there is a significant differential between the two readings, then the user can order further tests such as angiogram, CT scan, MRI or MA for example to determine if there is truly a problem. It is important to know where the blood pressure measurement is being taken because the readings vary as the distance from the patient's heart changes.

In general the farther away from the heart the blood pressure is taken, the narrower the vessels become and this effects the blood pressure readings. If you repeat the test at the same location each time, you have a better picture of the actual blood pressure rather than recording unintentional variations due to taking the readings at different positions. In practice, the primary care professional records the location of the blood pressure readings and if a problem is found, the location can be used to guide further specialists such as radiologists to guide other tests such as CT scans or MRI's to locate the pathology. The instant invention can provide important information to aide in a diagnosis long before a patient starts developing symptoms or complications at the primary care level.

Although the instant invention has been described in relation to particular embodiments thereof, many other variations and modifications and other uses will become apparent to those skilled in the art.

What is claimed is:

1. A device to hold multiple blood pressure reading units with position indicator comprising:
a blood pressure monitor holder;
said blood pressure monitor holder having a shelf portion a measurement anchoring collar;
a position indicator retractably disposed between said measurement anchoring collar and said blood pressure monitor;
said position indicator being adapted to display an approximate position of said dual input blood pressure monitor from a reference point wherein additional measurements are referenced and wherein a simultaneous measurement of both a left blood pressure reading and a right blood pressure reading are compared to provide diagnostic information.

2. The device to hold multiple blood pressure reading units with position indicator according to claim 1 wherein said position indicator is a flexible ruler.

3. The device to hold multiple blood pressure reading units with position indicator according to claim 1 further comprising a digital readout to indicate said approximate position.

4. The device to hold multiple blood pressure reading units with position indicator according to claim 1 further comprising a unit retainer attached to said blood pressure monitor holder adapted to removably secure said blood pressure reading units.
